(19) United States
(12) Patent Application Publication
(10) Pub. No.: US 2010/0311068 A1
(43) Pub. Date: Dec. 9, 2010

(54) ASSAY

(75) Inventors: Karin Mattsson, Sollentuna (SE); Kristina Domeika, Uppsala (SE)

Correspondence Address: MORRISON & FOERSTER LLP, 425 MARKET STREET, SAN FRANCISCO, CA, 94105-2482, US

(73) Assignee: BIOVATOR TECHNOLOGIES AB, Stockholm (SE)

(21) Appl. No.: 12/808,674

(22) PCT Filed: Dec. 18, 2008
(86) PCT No.: PCT/EP2008/067931

§ 371 Date: Jun. 16, 2010

(30) Foreign Application Priority Data
Dec. 18, 2007 (EP) PCT EP2007 064144
Sep. 23, 2008 (SE) 0802020-8

(51) Int. Cl. C12Q 1/68; G01N 33/68

(52) U.S. Cl. 435/6; 435/7.92

(57) ABSTRACT

The invention relates to process for in vitro prediction of a potentially allergenic substance wherein monocytes and/or macrophages and/or myelomonocytic cell lines are cultivated in the presence of the substance and interferon-γ, whereby productions of cytokines and/or neopterin are increased and measured. The allergic reaction may be estimated by measuring up regulated or down-regulated genes chosen from G1P2, OASL, IFIT1, TRIM22, IFI44L, MXI, RSAD2, IFIT3, IFITM1, IFIT2, C 33.28 HERV-H protein mRNA, IFITM3, XK, GPR15, MT1G, MT1B; MT1A, ADFP, IL8, MT1E, MT1F, MT1H, SLC30A1, SERPINB2, CD83, TncRNA or expression products from them are measured. The invention also relates to a reagent kit for use in the process comprising interferon-γ and reagents which recognise cytokines preferably each of IL-8 and neopterin and/or reagents such as probes recognising up-regulated or down-regulated genes.

The present invention relates to an improved Cytokine Profile Assay (CPA) and Gene Activation Profile Assay (GAPA) for in vitro prediction of a potentially allergenic substance, whereby monocytes and/or macrophages and/or myelomonocytic cell lines are cultivated in the presence of the substance and interferon-γ, and productions of cytokines and/or neopterin are increased and measured. The presence of cytokines may also be estimated by measuring cytokine associated genes or up regulated genes according to the invention.

It also relates to a reagent kit for performing the assay and the use of certain cell lines in the assay.

### PRIOR ART

The present Cytokine Profile Assay (CPA) is an in vitro test that allows the prediction of allergenic risk and adverse effects of substances. Substances intended for use as pharmaceuticals, food additives, cosmetic or hygienic products, industrial chemicals and other substances are analysed for their potential risk to induce an allergic reaction and other adverse reactions, such reactions may be skin irritating effects and toxic effects.

The analysis of the substances is performed on human monocytes and/or macrophages and/or myelomonocytic cell lines. No animals are involved since the substances are tested on human cells i.e. cells from the same species as they are intended for. Some substances may not have the same effect on different species, and tests performed on animals may give false results. Therefore the test is preferably performed with human cells.

The tests commercially used today are in vivo animal tests and on the account of ethical aspects there is a great demand of finding an in vitro method that can replace the currently used animal tests. Allergic reactions can be really serious for the person affected so there is a great demand from e.g. the pharmaceutical-, cosmetic- and the food industry to be able to identify these substances in an as early phase as possible.

In vitro evaluation of toxic effects on the immune system has been tested on splenocytes from mice (“In vitro evaluation of drug-induced toxic effects on the immune system as assessed by proliferative assays and cytokine production”, M. Pallardy et. al. Eur. Cytokine Net., Vol. 2 No 3, May-June 1991, pp. 201-206). The method was poorly effective detecting molecules inducing autoimmunity and hypersensitivity.

Previous studies have shown that neopterin and interleukin-8 (IL-8), produced by human blood cells, may be reliable signal molecules to identify allergenic substances. This hypothesis that lead to a Swedish patent No. 506 533 (WO 97/16732) directed to an in vitro method for the identification of human allergens and T-lymphocyte antigens. In vitro cultured human cells are exposed to the substance to be tested, and reactions associated with hyper sensitivity are measured. Stimulating cells with substances with known association with allergic reactions have been shown to initiate the release of certain cellular substances, mainly cytokines. The method covered by this patent was named cytokine profile assay (CPA). The concept of this test is that allergenic substances are able to induce specific patterns of neopterin and IL-8 production, measured in the supernatant of cultivated human peripheral blood mononuclear cells (PBMC). Analysis of the cytokine profile will specify whether there is an allergic reaction of type I or Type IV, irritation or no reaction at all.

Further validation studies of the CPA lead to the preferable use of a human monocyte cell-line as a reference system. Also, the method appeared most suitable to identify proteins known to induce type I allergy.

The method was later refined by measuring genes up regulated by a potentially allergenic or tissue irritating substance in PCT/SE2006/050336. This method is called gene activation profile assay, GAPA. GAPA also regards use of the expression products from one or more of the genes for in vitro analysis of allergy or tissue irritation.

### SUMMARY OF THE INVENTION

It has now turned out that when monocytes and/or macrophages and/or myelomonocytic cell lines are cultivated in the presence of a potentially allergenic substance and interferon-γ, the productions of cytokines and/or neopterin are increased in the presence of test substances having known association with hyper sensitivity. The effect seems even to be synergistic compared to when the cells are cultivated in the presence of a potentially allergenic substance and interferon-γ respectively.

The present invention relates to Cytokine Profile Assay (CPA) for in vitro prediction of a potentially allergenic substance, whereby monocytes and/or macrophages and/or myelomonocytic cell lines are cultivated in the presence of the substance and interferon-γ, and productions of cytokines and/or neopterin are increased and measured. The presence of cytokines may also be estimated by measuring cytokine genes or up regulated genes according to the invention.

In a further aspect, the invention relates to the Gene Activation Profile Assay (GAPA) for in vitro prediction of a potentially allergenic substance, whereby monocytes and/or macrophages and/or myelomonocytic cell lines are cultivated in the presence of the substance and interferon-γ, wherein the allergenicity of the substance is estimated by measuring up-regulated or down-regulated genes chosen from G1P2, OASL, IFIT1, TRIM22, IFI44L, MXI, RSAD2, IFIT3, IFITM1, IFIT2, SPR, GNB2, C 33.28 HERV-H protein mRNA, IFITM3, XK, GPR15, MT1G, MT1B; MT1A, ADFP, IL8, MT1E, MT1F, MT1H, SLC30A1, SERPINB2, CD83, CD86, TncRNA or expression products from them are measured.

It also relates to a reagent kit for performing the above processes, comprising interferon-γ and reagents which recognise cytokines preferably each of IL-8 and neopterin and/or reagents such as probes recognising cytokine genes or up regulated genes, and optionally further parts such as test cells, cell lines selected from the group consisting of MonoMac-6, THP-1, MUTZ-3, WBC264-9C and AML-193, a cell culture medium suitable for culturing the cell line, antibiotics, a positive control, a negative control, a culture plate or flask, and/or instructions describing the method to be performed.

In a further embodiment, the invention relates to the use of monocytes and/or macrophages and/or myelomonocytic cell lines in the methods according to the invention. Cell lines presently contemplated for use in the methods according to the invention are MonoMac-6, THP-1, MUTZ-3, WBC264-9C and AML-193, as described below.

The method will be offered as an alternative to animal tests for food additives, cosmetic or hygienic products, pharmaceuticals, industrial chemical, drugs and other substances where adverse reactions are to be avoided.

### FIGURE LEGENDS

The invention is illustrated with the following figures.

FIG. 1: MonoMac-6 cells were stimulated for 24 h with for the native birch allergen Bet v 1 and human serum albumin (HSA) at 0.2 to 20 μg/ml. Dose response curves are shown in the figure. The substances were stimulated with (filled points) or without (open points) IFN-γ in the cell culture medium during stimulation. The cells were cultured in RPMI+10% FCS. Each point represents the mean of triplicate cultures. The standard deviation within triplicate cultures is indicated with error bars.

FIG. 2a: Effect of IFN-γ on the cellular neopterin-response towards protein allergens. MM6-cells were stimulated for 24 hrs with allergen proteins Bet v1 or Ara h2, as wells as with control substances, in the presence of IFN-γ (100 U/ml) (▪) or absence of IFN-γ ( [symbol] ). The allergens were tested in concentrations ranging from 0.24 to 20 μg/ml. Dose responses are indicated with broken lines. Each bar represents the mean of triplicate cultures. The standard deviation within triplicate cultures is indicated with error bars. MonoMac-6 cells were cultured in RPMI supplemented with 10% FCS.

FIG. 2b: Effect of IFN-γ on the cellular neopterin-response towards protein with lack of considerably allergenicity. MM6-cells were stimulated for 24 hrs with proteins HSA, Lectin sol. tub. or gelatin, in the presence of IFN-γ (100 U/ml) (▪) or absence of IFN-γ ( [symbol] ). The proteins were tested in concentrations ranging from 0.24 to 20 μg/ml. Each bar represents the mean of triplicate cultures. The standard deviation within triplicate cultures is indicated with error bars. MonoMac-6 cells were cultured in RPMI supplemented with 10% FCS.

FIG. 3a: Cellular neopterin levels were increased in a dose dependent manner towards protein allergens. MM6-cells were stimulated for 24 hrs with allergen proteins α-Amylase aspergillus, Ara h2, Alt a 1 or Phl p 1 as wells as with control substance (HSA), in the presence of IFN-γ (100 U/ml). The allergens were tested in concentrations ranging from 0.7 to 60 μg/ml. Dose responses are indicated with broken lines. Each bar represents the mean of triplicate cultures. The standard deviation within triplicate cultures is indicated with bars. MonoMac-6 cells were cultured in RPMI supplemented with 10% FCS.

FIG. 3b: Cellular neopterin levels were increased in a dose dependent manner towards protein allergens. MM6-cells were stimulated, in the presence of IFN-γ, for 24 hrs with allergen proteins Cor a 8, Amb a 1 or with control substance Lens lectin (LCA). The proteins were tested in concentrations ranging from 0.7 to 60 μg/ml. Dose responses are indicated with broken lines. Each bar represents the mean of triplicate cultures. The standard deviation within triplicate cultures is indicated with error bars. MonoMac-6 cells were cultured in RPMI supplemented with 10% FCS.

FIG. 3c: Weak cellular neopterin response upon stimulation with proteins having low potential of being allergenic. MM6-cells were stimulated, in the presence of IFN-γ, for 24 hrs with proteins Termamyl or Soy lectin. The proteins were tested in concentrations ranging from 2.2 to 180 μg/ml. Dose responses are indicated with broken lines. Each bar represents the mean of triplicate cultures. The standard deviation within triplicate cultures is indicated with error bars. MonoMac-6 cells were cultured in RPMI supplemented with 10% FCS.

FIG. 4a: Effect of IFN-γ on the cellular neopterin-response towards chemical allergens. MM6-cells were stimulated for 24 hrs with chemicals substances associated with respiratory sensitization (HCPt, TMA or MDI) or with skin sensitization (DNCB) in the presence of IFN-γ (100 U/ml) (▪) or absence of IFN-γ [symbol] . The test concentrations are given in μg/ml). Each bar represents the mean of triplicate cultures. The standard deviation within triplicate cultures is indicated with error bars. MonoMac-6 cells were cultured in RPMI supplemented with 10% FCS. Bet v1 and HSA included as controls.

FIG. 4b: Cellular neopterin response upon stimulation with chemicals associated with skin sensitization. MM6-cells were stimulated, in the presence of IFN-γ, for 24 hrs with chemicals associated with skin sensitization. Test concentrations are given in μg/ml. Each bar represents the mean of triplicate cultures. The standard deviation within triplicate cultures is indicated with error bars. MonoMac-6 cells were cultured in RPMI supplemented with 10% FCS.

FIG. 4c: Cellular neopterin response upon stimulation with chemicals associated with skin sensitization or irritantancy. MM6-cells were stimulated, in the presence of IFN-γ, for 24 hrs with chemicals associated with skin sensitization or irritancy. Test concentrations are given in μg/ml or for glycerol in percentage. Each bar represents the mean of triplicate cultures. The standard deviation within triplicate cultures is indicated with error bars. MonoMac-6 cells were cultured in RPMI supplemented with 10% FCS.

FIG. 4d: Cellular neopterin response upon stimulation with chemicals associated with irritancy. MM6-cells were stimulated, in the presence of IFN-γ, for 24 hrs with chemicals associated with irritancy. Test concentrations are given in μg/ml. Each bar represents the mean of triplicate cultures. The standard deviation within triplicate cultures is indicated with error bars. MonoMac-6 cells were cultured in RPMI supplemented with 10% FCS.

FIG. 5a: Mono Mac-6 cells were stimulated in RPMI+10% FCS for 24 hrs with for the native peanut allergen Ara h 2 (20 and 6.6 microgram per ml) and human serum albumin (HSA; 6.6 microgram per ml). The substances were stimulated with IFN-γ in the cell culture medium during stimulation. At each experiment a number of wells with unstimulated cells were included and the results are also shown in the graph. For each experiment the results are shown as mean of triplicate cultures. The standard deviation of the triplicates is indicated with bars.

FIG. 5b: MonoMac 6 cells were stimulated in the serum free media Panserin 411 for 24 hrs with for the native peanut allergen Ara h 2 (20 and 6.6 microgram per ml) and human serum albumin (HSA; 6.6 microgram per ml). The substances were stimulated with IFN-γ in the cell culture medium during stimulation. At each experiment a number of wells with unstimulated cells were included and the results are also shown in the graph. For each experiment the results are shown as mean of triplicate cultures. The standard deviation of the triplicates is indicated with bars.

FIG. 5c: MonoMac 6 cells were stimulated in the serum free media Panserin 411 for 48 hrs with for the native peanut allergen Ara h 2 (20 and 6.6 microgram per ml) and human serum albumin (HSA; 6.6 microgram per ml). The substances were stimulated with IFN-γ in the cell culture medium during stimulation. At each experiment a number of wells with unstimulated cells were included and the results are also shown in the graph. For each experiment the results are shown as mean of triplicate cultures. The standard deviation of the triplicates is indicated with bars.

FIG. 6a. Cellular neopterin levels were increased in a dose dependent manner towards protein allergens in cells cultured in serum free media. MM6-cells, cultured in Panserin cell culture media were stimulated for 24 hrs with allergen proteins Ara h2, Cor a 8, Alt a 1 or Phl p 1 as wells as with control substance (HSA), in the presence of IFN-γ(100 U/ml). The allergens were tested in concentrations ranging from 0.7 to 60 μg/ml. Each bar represents the mean of triplicate cultures. The standard deviation within triplicate cultures is indicated with error bars.

FIG. 6b: Cellular neopterin levels were increased in a dose dependent manner towards protein allergens in cells cultured in serum free media. MM6-cells, cultured in Panserin cell culture media were stimulated for 24 hrs with allergen proteins Ara h2 or Amb a 1 as wells as with non-allergenic gelatine or control substance (HSA), in the presence of IFN-γ(100 U/ml). Each bar represents the mean of triplicate cultures. The standard deviation within triplicate cultures is indicated with error bars.

FIG. 6c: Cellular neopterin levels were increased in a dose dependent manner towards protein allergens in cells cultured in serum free media. MM6-cells, cultured in Panserin cell culture media were stimulated for 24 hrs with allergen proteins Ara h2, Gal d 2 (Ovalbumin) or Gal d 3 (Conalbumin) as wells as with non-allergenic insulin or control substance (HSA), in the presence of IFN-γ(100 U/ml). Each bar represents the mean of triplicate cultures. The standard deviation within triplicate cultures is indicated with error bars.

FIG. 6d: Weak cellular neopterin response upon stimulation with proteins having low potential of being allergenic. MM6-cells, cultured in Panserin cell culture media were stimulated for 24 hrs with allergen proteins Ara h2, Amb a 1, as wells as with proteins of low probability of being allergenic, lectin soluanum tuburosum or control substance (HSA), in the presence of IFN-γ(100 U/ml). Each bar represents the mean of triplicate cultures. The standard deviation within triplicate cultures is indicated with error bars.

FIG. 7a: Cellular neopterin levels were increased in a dose dependent manner towards protein allergens in cells cultured in serum free media. MM6-cells, cultured in Panserin cell culture media were stimulated for 48 hrs with allergen proteins Ara h2, Cor a 8, Alt a 1 or Phl p 1 as wells as with control substance (HSA), in the presence of IFN-γ(100 U/ml). The allergens were tested in concentrations ranging from 0.7 to 60 μg/ml. Each bar represents the mean of triplicate cultures. The standard deviation within triplicate cultures is indicated with error bars.

FIG. 7b: Cellular neopterin levels were increased in a dose dependent manner towards protein allergens in cells cultured in serum free media. MM6-cells, cultured in Panserin cell culture media were stimulated for 48 hrs with allergen proteins Ara h2, Amb a 1, α-amylase aspergillus or Gal d 2 as wells as with control substances gelatine, lectin solanum tuburosum or HSA, in the presence of IFN-γ(100 U/ml). Each bar represents the mean of triplicate cultures. The standard deviation within triplicate cultures is indicated with error bars.

FIG. 7c: Cellular neopterin levels were increased in a dose dependent manner towards protein allergens in cells cultured in serum free media. MM6-cells, cultured in Panserin cell culture media were stimulated for 48 hrs with allergen proteins Ara h2, Gal d 2 (Ovalbumin), Gal d 3 (Conalbumin) or with control substance (HSA) or insulin, in the presence of IFN-γ(100 U/ml). Each bar represents the mean of triplicate cultures. The standard deviation within triplicate cultures is indicated with error bars.

FIG. 8: THP-1 cells exposed to protein allergen Ara h2 and LPS as well as to HSA at different test concentrations for 24 hrs. Cells are culture in RPMI supplemented with 10% FCS.

FIG. 9: Overview of the main procedure steps of typical embodiments of the Cytokine Profile Assay and the Gene Activation Profile Assay.

### DETAILED DESCRIPTION

The process according to the invention is for in vitro prediction of a potentially allergenic substance. Monocytes and/or macrophages and/or myelomonocytic cell lines or other equivalent cell lines having monocytic, macrophagic and/or myelomonocytic origin are cultivated in the presence of the substance and interferon-γ, whereby productions of cytokines and/or neopterin are increased and measured. The substance to be tested and interferon-γ may be added mixed together or at the same time. Alternatively the substance to be tested may be added before the interferon-γ or vice versa.

It has turned out that especially the production of neopterin is increased when interferon-γ is added.

Neopterin (6-D-erythrotrihydroxypropyl-pterin), is a substance of low molecular mass, that becomes biosynthesized from guanosinetriphosphate (GTP) by GTP-cyclohydrolase I, the key enzyme of pteridine-biosynthesis. It is formed and released by human monocytes/macrophages in vivo and by some myelomonocytic cell lines in vitro (such as THP-1 and MM6). Neopterin production reflects the stage of activation of the cellular immune system. Interferon-γ (IFN-γ) is mainly produced by activated T-lymphocytes (especially of so-called TH-1-type cells) and it is recognized as the only cytokine that significantly induces production of neopterin. The level of neopterin formation in vivo correlates with the IFN-γ influence on monocytes/macrophages.

Applicants previous studies have shown that neopterin release is specifically up-regulated when cells are stimulated with allergen extracts (Type I associated allergens).

The presence of one or more cytokines chosen from IL-1, IL-1β, IL-2, IL-4, IL-5, IL-6, IL8 IL-10, IL-12, TNF-α and IFN-γ, is an indication of class IV cell mediated T-cells immunity and delayed type hypersensitivity such as cellular immunity, delayed allergy and contact eczema.

The presence of one or more cytokines chosen from IL-1, IL-1β, IL-2, IL-4, IL-5, IL-6, IL8 IL-10, IL-12, TNF-α and IFN-γ and/or the presence of high levels of neopterin, is an indication class I immune response type from T and B lymphocytes and inflammatory cells and immediate type hypersensitivity such as asthma, hay fever, urticaria and rhinitis.

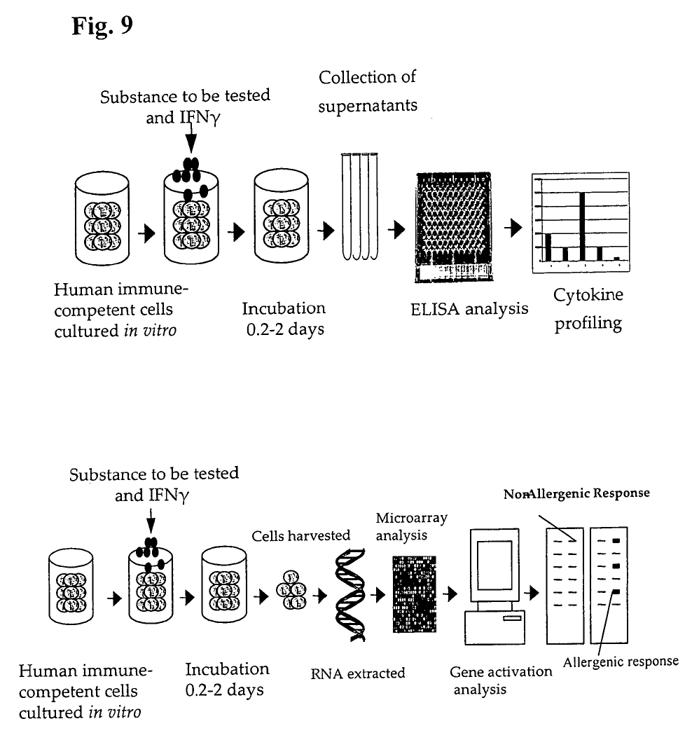

The method may be performed as outlined in FIG. 9. Briefly human cells are cultured in 96-well plates. The test substances and IFN-γ are added to the cells. The cells are incubated e.g. for at least 5 hours such as from 5 to 48 hours, e.g. from 12 to 36 hours especially from 20 to 24 hours. The supernatants are analysed. This may be done by collecting them and transferring them to a 96-well-ELISA plate. Then the cytokine production is measured. This may be done by measuring the cytokines as such. According to one embodiment ELISA analysis of cytokines in supernatants are performed. Especially neopterin is measured. Thereafter the test results are evaluated. For each substance, a dose response curve may be established. The allergic reaction may also be measured by detection of up-regulated or down-regulated genes: by measurement of RNA or DNA extracted from the cells, i.e. by determining a gene activation profile.

According to a further embodiment cytokines of class IV type connected with cell mediated T-cells immunity chosen from IL-2, IL-8, IL-10, IFN-gamma, IL-4, IL-5, and soluble products such as sCD8 and sIL-2R are also measured.

According to still another embodiment cytokines of class I type and an immune response type from T and B-lymphocytes and inflammatory chosen from IFN-gamma, IL-2, IL-10, IL-4, IL-5 and soluble products such as sCD8 and sIL-2R are also measured.

According to the invention it is also possible to estimate the allergic reaction by measuring up-regulated or down-regulated genes chosen from: G1P2, OASL, IFIT1, TRIM22, IFI44L, MXI, RSAD2, IFIT3, IFITM1, IFIT2, SPR, GNB2, C 33.28 HERV-H protein mRNA, IFITM3, XK, GPR15, MT1G, MT1B; MT1A, ADFP, IL8, MT1E, MT1F, MT1H, SLC30A1, SERPINB2, CD83, TncRNA or expression products from them. In the perspective the expression of one or more of G1P2, OASL, IFIT1, TRIM22, IFI44L, MXI, RSAD2, IFIT3, IFITM1, IFIT 2, indicates Type I allergy; one or more of SPR, GNB2, XK, IFITM3, indicates non allergy; one or more of C 33.28 HERV-H protein mRNA, IFITM3, XK, GPR15, indicates TYPE I/IV haptenes and one or more of MT1G, MT1B; MT1A, ADFP, IL8, MT1E, MT1F, XK, IFITM3, MT1H, SLC30A1, SERPINB2, GNB2, MTIB, CD83, TncRNA genes indicates Type IV allergy.

We classify adverse tissue reactions to foreign substances as follows.

Class I: Allergic immune reaction type I. This is also named immediate type hypersensitivity and is mediated by IgE anti-bodies produced specifically to the foreign substance. An acute inflammatory reaction is produced, histamine is often produced and examples of symptoms are asthma hay fever, urticaria and rhinitis. This type requires that the substance can induce a fully mature immune response where all components of the immune system participate. This is also considered as a final step immune reaction.

Class IV: Inflammatory immune reaction type IV. This is also named delayed type hypersensitivity and is mediated by sensitised T lymphoccytes type TH-1, considered as a first step type immune reaction. Allergic contact dermatitis is an example of this type of reaction.

For Class I and Class IV see “Immunology” by Ivan Roitt, Jonathan Brostoff and David Male, Gower Medical Publishing London—New York, 1989, page 19.1-19.20 and 22.1-22.10, which is incorporated as a reference.

Three main types of cytokine profiles have been identified.

Class 0 type: Secretion of alarm cytokines indicating damage to connective tissue, fibroblasts, endothelial cells, epithelial cells and unspecific inflammatory white blood cells. Members of this group are IL-1, IL-6, IL-12 and TNF.

Class I type: Secretion of cytokines of an immune reponse type from lymphocytes and inflammatory cells. These includes cytokines from Class 0 and in addition IFN-gamma, Neopterin, IL-2 and IL-10. Theoretically as known from animal in vivo studies also IL-4 and IL-5 should be produced here, but these substances are notoriously difficult to determine and are therefore not routinely included in our test protocolls.

Class IV type: Secretion of the cytokines of Class 0 unspecific type and in addition IL-2, IL-8, IL-10 and IFN-gamma.

The complete final type immune response Class I drives to Neopterin production whereas the Class IV primary type immune response does not stimulate the immune system as long as to Neopterin production.

The cytokines listed above are only preferably selected representative members. In addition other cytokines may be used as well and the patent is claimed for the use of analysis of all substances listed in The Cytokine Facts Book, Eds Callard R. E. and Gearing, A. J. H., Academic Press 1994 hereby incorporated as a reference and future updated issues to predict the grade of maturity of the response that a substance has the potential to evoke.

Examples of interleukins are IL-1, IL-2, IL-3, IL-4, IL-5, IL-6, IL-7, IL-8, IL-9, IL-10, IL-11, IL-12, IL-13, IL-14, IL-15. Examples of other cytokines (in alphabetical order) BDNF, CNTF, EGF, Epo, FGF, G-CSF, GM-CSF, I-309/TCA-3, yIP-10, IFNα, IFNβ, IFNγ, LIF, LT(TNFβ), MCP-1, 2 and 3, M-CSF, MIF, MIP-1α, MIP-1β, MIP-2, NGF, NT-3, NT-4, OSM, PBP, PBSF, PDGF, PF-4, RANTES, SCF, TGFα, TGFβ, TNFα, Tpo, VEGF

According to the invention the presence of inflammatory immune reaction type IV is preferably analysed by using IL-8. It has turned out that IL-8 is developed and is present in raised levels during type IV reactions.

Neopterin being present in higher amounts when type I reaction is at hand, IL-8 and/or neopterin can especially be used to distinguish between the inflammatory immune reaction type IV and the allergic immune reaction type I.

Based on the outcome of the in vitro test, the cytokine pattern induced by a substance under investigation, a prediction will be made regarding the potential of the substance to cause adverse reaction if humans or animals are exposed to it.

Antibiotics may be added to prevent the growing of unwanted micro-organisms. Penicillin and streptomycin may be added in concentrations of 25-100 U/ml.

Mitogens or substances with known effects on the immune system can be used as positive controls to give better results. Any mitogen as mentioned in Daniel P. Stites: Clinical Laboratory Methods for Detection of Antigens & Antibodies in Basic and Clinical Immunology, Lange Medical Publications, Los Altos Calif., 1984 may be used. This reference is incorporated in this description by reference.

Phytohemagglutinin (PHA-L) is preferably used and dissolved in a suitable medium to a final concentration of in the wells of 250 μg per ml.

The medium may be any medium used for cultivation of cells, especially human cells and cells derive from human blood. RPMI 1640 is one example of suitable medium. The medium may also be a serum free medium, such as Panserin 411.

The test can be performed in tubes or preferably in the wells of microtiter plates.

According to one embodiment of the invention the highest concentration of the substance being non toxic to the monocytes, macrophages and/or myelomonocytic cell lines may be serial diluted.

For substances to be tested for effects in the system, different concentrations are added to the cells and incubated at about 37° C. The highest concentration of the substance being non toxic to the cells indicated by e.g. inspection in microscope in presence of vital stain e.g. trypan blue, propidium iodide, or any other viability test may be used as start concentration. Serial dilution of the substance is then performed. In cases where the substance is diluted in RPMI 1640 the appropriate control is medium only. When other diluents are used, controls of the appropriate diluent at corresponding concentrations are used.

In one embodiment of the invention the substances to be tested are proteins. If the substances to be tested are potential allergens, the potential allergens to be tested can be tested as component(s) of extracts or in pure native form, either isolated from an allergen source or recombinantly produced.

### Cytokine Assays

The plates are removed from the incubator at various time intervals, and cytokines released from the cultured cells are measured in the supernatants. Supernatants can either be tested immediately or stored at −20° C. until tested. For cytokine determinations diagnostic kits of various origins are used. Patent is claimed for this procedure using any available or newly constructed test using cytokine quantifying including bioassays, immunoassays or chemical assays or other.

The instructions of the manufacturers are followed. All these tests are enzyme immunoassays (EIA:s). The principle is that microtiter plate wells are labelled with a cytokine specific capture antibody. If cytokine is present in the samples added to the wells it will be captured to the bottom of the well. To quantify how much cytokine that has been captured a second antibody, labelled with an enzyme may be added to the wells. The reaction may thereafter be measured as colour developed after addition of a substrate for the enzyme. The values for the test wells may be compared with a standard curves obtained from a series of known amounts of cytokine. A dose response curve may be established for each substance.

The invention also concerns a kit comprising one or more reagents recognizing the cytokines of class I type, and/or the cytokines of class IV. Examples of such cytokines are the ones mentioned in this description. Reagents reacting for the presence of neopterin and IL-8 are preferred.

These reagents may be antibodies or any other reagents that are sensitive for these substances. The reagents may be supported by a carrier such as a strip, titre plates, microtiter plates, ELISA plates, test tubes etc. The carriers may be of different sizes. The carrier material may be any solid or semi-solid material that does not interfere with the reaction between the reagent and the cytokine. The carrier can take on a variety of shapes and compositions, including micro particles, beads, porous and impermeable strips and membranes, the interior surface of reaction vessels such as test tubes and microtiter plates, and the like. Microtiter plates and beads may be of plastic, such as styrene or acryl-polymers or glass. Nitrocellulose can be used, preferably in the form of filters, strips or discs. Means for attaching a desired reaction partner to a selected solid support will be a matter of routine skill to the worker in the field. It is also possible to use flow cytometer.

The following commercial kits (with stated manufacures) may be used:

IL-1beta: Immunotech; Chromgenix

### IL-2: Immunotech; Chromgenix

### IL-4: R&D Systems Neopterin: Henning Berlin, IBL-Hamburg Neopterin ELISA

### IL-6: Immunotech, Chromgenix

IL-8: Assay Res. Inc

### Interferon-gamma: Genzyme

sCD8: T Cell Diagnostics

### SIL-2R Immunotech

### TNF: Medgenix

### IL-10: Medgenix

The instructions of the manufacturers were followed. All these tests are enzyme immunoassays (EIA:s). The principle is that microtiter plate wells are labelled with a cytokine specific capture antibody. If cytokine is present in the samples added to the wells it will be captured to the bottom of the well. To quantify how much cytokine that has been captured a second antibody, labelled with an enzyme is added to the wells. The reaction is thereafter measured as colour developed after addition of a substrate for the enzyme. The values for the test wells are compared with a standard curve obtained by adding a series of known amounts of cytokine. The values obtained for control wells with no substance added are used as background values for cytokine levels.

### Gene Activation Assays

Detection of up-regulated or down-regulated genes may be performed as described in PCT/SE2006/050336. For genes with gene products expressed on the surface of cells it is also possible to detect up-regulation or down-regulation by FACS.

### Reagent Kits

The invention further relates to a reagent kit for use in the process according to the invention, characterized in that it comprises interferon-γ and reagents which recognize cytokines IL-8 and neopterin and/or reagents such as probes recognizing up-regulated or down-regulated genes.

The reagent kit may accordingly comprise probes that recognize products produced during the expression of any of G1P2, OASL, IFIT1, TRIM22, IFI44L, MXI, RSAD2, IFIT3, IFITM1, IFIT2, SPR, GNB2, XK, IFITM3, C 33.28 HERV-H protein mRNA, IFITM3, XK, GPR15, MT1G, MT1B; MT1A, ADFP, IL8, MT1E, MT1F, MT1H, SLC30A1, SERPINB2, CD83, TncRNA. Such probes may be DNA or RNA probes, or fluorescent labeled molecules binding specifically to expression products displayed on the cell surface of the test cells for use in an analysis by FACS.

The reagent kit may further also comprise test cells chosen from monocytes and/or macrophages and/or myelomonocytic cell lines.

Throughout this specification and the claims which follow, unless the context requires otherwise, the word “comprise”, or variations such as “comprises” or “comprising”, will be understood to imply the inclusion of a stated component or group of components but not the exclusion of any other components or group of components.

All publication mentioned herein are hereby incorporated as reference. The invention will now be described by the following non-limiting examples.

### Experimental

The invention is further illustrated by the following experiments showing some embodiments of the invention. The given experiments shall not be construed as limiting the scope of the invention, which is that of the appended claims.

### Materials and Methods

### Cell Culture

The myelomonocytic cell line MonoMac-6 (obtained from DSMZ, accession number ACC 124, Prof. H. W. L. Ziegler-Heitbrock, Inst. For Immunology, University of Munich, Munich) was cultured in cell culture flasks (Corning). Cells were cultured in RPMI 1640 medium (Gibco BRL), containing 20 mM HEPES and L-glutamine and supplemented with 10% heat-inactivated foetal bovine serum (Gibco BRL), penicillin (100 U/mL; Sigma-Aldrich), streptomycin (100 μg/mL; Sigma-Aldrich), sodium pyruvate (1.0 mM; Gibco BRL), human insulin (9 μg/mL; Gibco BRL) and non-essential amino acids (Gibco BRL) or cultured in Panserin 411 (Pan Biotech) supplemented with penicillin (100 U/mL), streptomycin (100 μg/mL).

The acute monocytic leukaemia cell line THP-1 (ATCC, USA, accession number TIB-202) was cultured in RPMI 1640 medium with 10 mM HEPES (Gibco BRL). Addition of 4 mM L-glutamine (Gibco BRL), 1.5 g/L sodium bicarbonate, 4.5 g/L glucose (Gibco BRL), 1.0 mM sodium pyruvate (Gibco BRL), 0.05 mM 2-mercaptoethanol (Gibco BRL), 10% fetal bovine serum (FBS) (Gibco BRL), 100 U/ml penicillin and 100 μg/ml streptomycin (Sigma-Aldrich).

Other cell lines that are contemplated for use in the present invention are WBC264-9C (ATCC accession number HB-8902), AML-193 (DSMZ accession number ACC 549), MUTZ-3 (DSMZ accession number ACC 295). Information on suitable culture conditions for these cell lines are available from ATCC and DSMZ, respectively.

### Stimulation Procedure

The cells were split approximately 1:3 the day before stimulation. The stimulation procedure was performed in three steps. The first step was pre-dilution of test substances, to double the final concentration, in cell culture medium. Furthermore, Interferon-γ (IFN-γ) (100 U, PreproTech) was added in the cell culture media for 24 or 48 hours during stimulation of cells with test substances. The number of cells was then determined by trypan blue exclusion and the cells were diluted to twice the final test concentration (2×106 cells/mL) if not otherwise indicated and 100 μl of cell suspension was added per well in 96 well tissue culture plates (Corning). Finally, a 100 μl of pre-diluted test substance (triplicate cultures) was added to the tissue culture plates. On each cell culture plate a protein purified from birch extract (Bet v1; Phadia) was added as positive control and HSA was added as negative control. The stimulated cells were cultured at 37° C., 5% CO₂, for 24 or 48 hours. The cell suspension was transferred to new cell culture plates and stored at −20° C. until analyzed. Thereafter the cell suspension was analyzed for presence of neopterin by ELISA (IBL-Hamburg) following the manufacturers instructions.

### Test Substances

Ammonium hexachloroplatinate (HCPt) (Sigma Aldrich CAS No. 16919-58-7), Trimellitic anhydride (TMA) (Sigma Aldrich CAS No. 552-30-7), Diphenylmethane diisocyanate (MDI) Diphenylmethane diisocyanate (MDI) (Sigma Aldrich, CAS No. 101-68-8), 2,4 Dinitrocholorobenzene (Sigma Aldrich, CAS No. 97-00-7), propyl gallate (PLG) (Sigma Aldrich, CAS No. 121-79-9), Isoeugenol (Sigma Aldrich, CAS No. 97-54-1), Benzocaine (Sigma Aldrich, CAS No. 94-09-7), Penicillin G (Sigma Aldrich, CAS No. 69-57-8), Nickel sulphate (Sigma Aldrich, CAS No. 10101-97-0), Methyl salicylate (Sigma Aldrich, CAS No. 119-36-8), Sodium lauryl sulphate (SLS) (Sigma Aldrich, CAS No. 151-21-3), Glycerol (Sigma Aldrich, CAS No. 56-81-5), Salicyl Acid (SA) (Sigma Aldrich, CAS No. 69-72-7), p-Aminobenzoic acid (PABA) (Sigma Aldrich, CAS No. 150-13-0), Alpha-amylase, from Aspergillus oryzae (Sigma Aldrich, CAS No. 9001-19-8), Human albumin (Sigma Aldrich, CAS No. 70024-90-7), Lipxygenase (soya been seed), (Sigma Aldrich, L7395L), Insulin (Gibco BRL), Termamyl (Sigma Aldrich, CAS No. 9000-85-5), Tropomyocin (vertebrate) (Sigma Aldrich, CAS No. 9067-56-5), Alpha-amylase (human saliva) (Sigma Aldrich, CAS No. 9000-90-2), Lectin: arachis hypogaea lectin (PNA) (peanut) (Medicago, 05-0116), Lectin: glycine max (soy been) (Medicago, 05-0117), Lectin: solanum tuberosum (potato) (Sigma Aldrich, L4266), D-ribulose 1,5-diphosphate carboxylase (RUBISCO) (Sigma Aldrich, CAS No. 9027-23-0), Gelatin (Sigma Aldrich, CAS No. 9000-70-8), Lectin: lens culinaris (LCA/LCH) (lens) (Medicago, 05-0104), LAL standard (Control Std Endotoxin (CSE)) (Endosafe KTA) and Lipopolysaccharide (LPS) (Sigma Aldrich, L3880-100MG). Native components from Phadia: Art v 1 (Mugworth), Ara h 2 (Peanut), Bet v 1 (Birch), Cor a 8 (LTP, Hazelnut), Alt a 1 (Alternaria), Phl p 1 (Thimothy), Amb a 1 (Ragweed), Gal d 2 (Ovalbumin, Egg white) and Gal d 3 (Conalbumin, Egg, white). Test concentrations are given in Figures. The test concentrations used were chosen based on data from optimization experiments performed previously. Viability of cells should not be below 80%.

### Cell Viability: Measurement by Propidium Iodide (PI) Staining and Analysis by Flow Cytometry

Monitoring of cell viability was made in each experiment by staining with 0.4% trypan-blue (1:1) and cell counting using a haemocytometer: Dead cells are stained with trypan blue. % Viable Cells=number of living cells×100/total number of cells. In order to find relevant test concentrations (in previous optimization studies) cell viability was determined using propidium iodide (PI) staining and flowcytometry analyses. The cells were re-suspended in 1 ml ice cold PBS (SVA) and centrifuged at 300 g for five minutes and then suspended in 0.2 ml ice cold PBS supplemented with 8 μl PI staining solution (Becton Dickinson). The cells were then transferred to FACS tubes and stored on ice in the dark until analysis with a FACScan flow cytometer and CellQuest software (Becton Dickinson) within one hour. Dead control cells were made by freezing and thawing non-induced control cells in liquid nitrogen three times. A number of 50.000 cells were analyzed from each sample. Based on light scatter characteristics an electronic gate (R1) was used to exclude signals derived from cell debris. The electronic gate for calculation of the number of dead cells was set using the freeze/thawing control cells.

### Results

Presence of IFN-γ Significantly Increased the Levels of Neopterin Induced by Bet v Land Ara h 2 but not Levels Produced in Cells Stimulated with Control Substances.

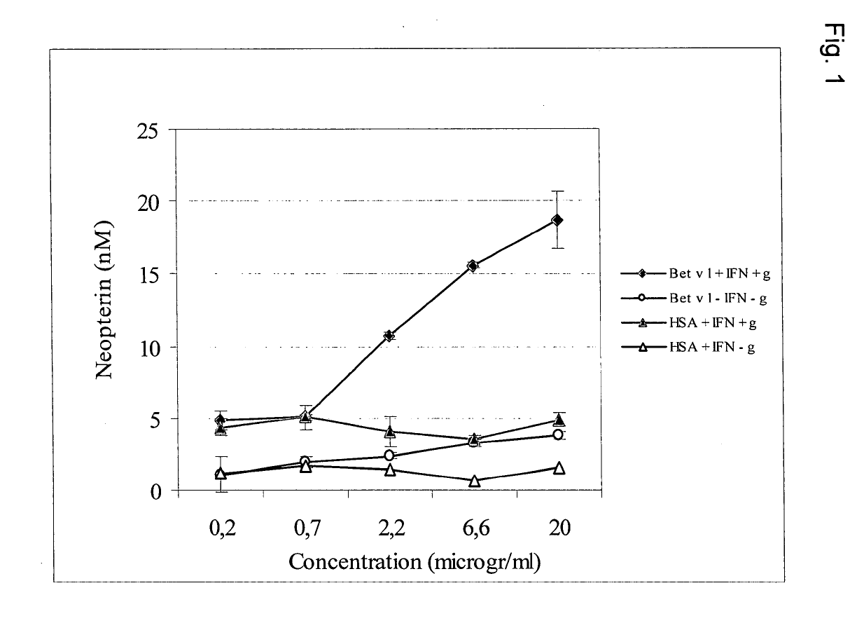

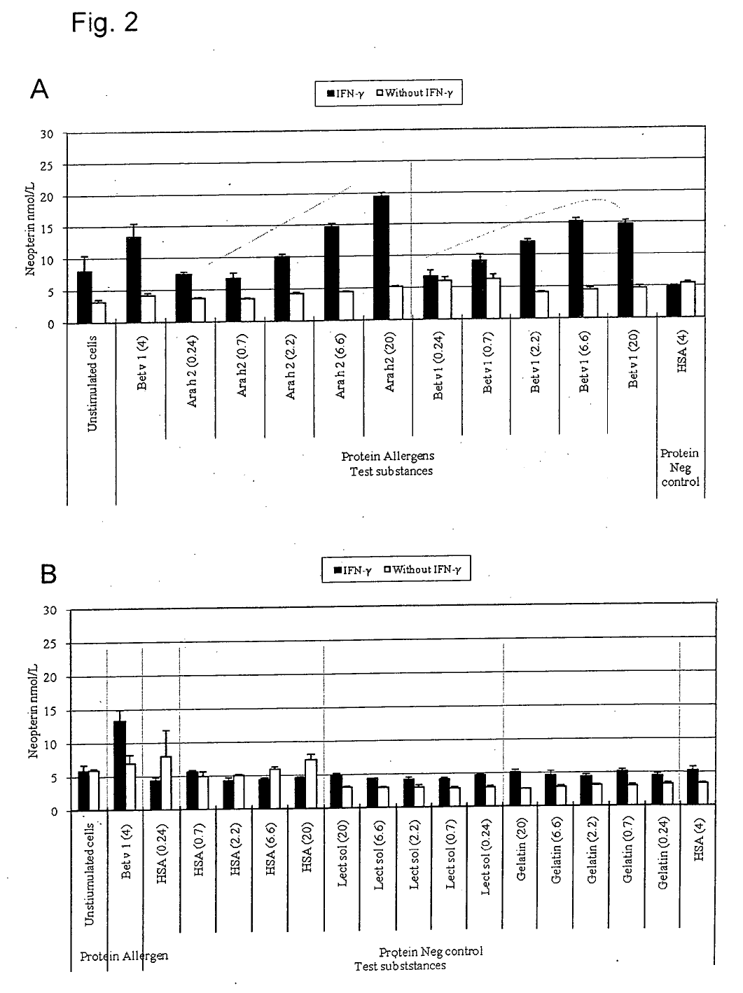

Stimulation of MM6-cells cultured in RPMI+10% FCS with native protein allergens induces neopterin levels of approximately 5 nmol/l. To determine if co-stimulation with IFN-γ could specifically increase the neopterin levels produced at induction with the protein allergens, the cells were stimulated with and without presence of IFN-γ (100 U/ml) during stimulation. MonoMac-6 cells were at a cell concentration of 1×10⁶ cells per ml for 24 h (FIG. 1). In the presence of IFN-γ the Bet v 1 (2.2-20 μg/ml) induced a 3-fold increase in neopterin production compared to the neopterin production obtained in cells stimulated with Bet v 1 only. Cells exposed to control substance HSA (2.2-20 μg/ml) in the presence of IFN-γ, increase the neopterin production less than 1 fold change. In addition, stimulation with native allergen Ara h 2 (20 μg/ml) showed a 3-fold increase in neopterin production in the presence of IFN-γ compared to cells only with Ara h 2 (FIG. 2a). Co-stimulation with Bet v1 and IFN-γ or Ara h 2 and IFN-γ showed a neopterin production in a dose response manner. Stimulation with IFN-γ in combination with HSA, lectin solanum tuburosum or gelatin (substances considered to lack considerably allergenicity) did not induce any neopterin production above the background levels (FIG. 2b). The cell viability upon stimulation with protein allergens was unchanged (at all test concentrations) compared to cells stimulated with IFN-γ only or cells cultured in media only (data not shown).

Neopterin Levels were Increased in a Dose Dependent Manner Towards Protein Allergens.

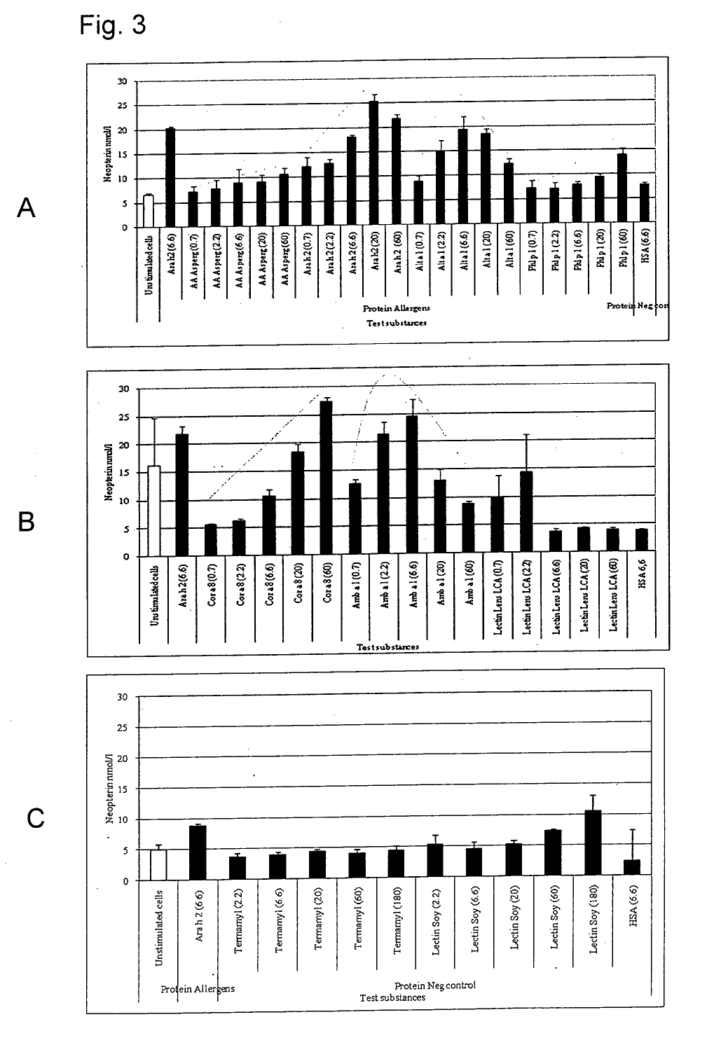

The CPA test protocol, optimised using IFN-γ as co-stimulator, was then further evaluated using an extended test panel covering protein allergens (FIG. 3a and FIG. 3b) or proteins with less or no allergenic potential (FIG. 3b and FIG. 3c). The protein allergens were tested in concentrations ranging from 0.2-180 μg/ml, based on earlier pilot studies aiming at finding relevant test concentrations (data not shown). All allergenic substances induced increased neopterin production in a dose dependent manner (eight out of eight). Proteins associated with low or little allergenicity such as HSA, termamyl or soy lectin did not induce neopterin above background levels (un-stimulated cells). Soy lectin gave a neopterin response at a test concentration of 180 μg/ml, comparable to the cellular response towards Ara h 2 at 6.6 μg/ml. Soy lectin at a test concentration of 6.6 μg/ml did not induce any neopterin response above the levels of control cells (Unstimulated cells or cells stimulated with HSA (6.6 g/ml).

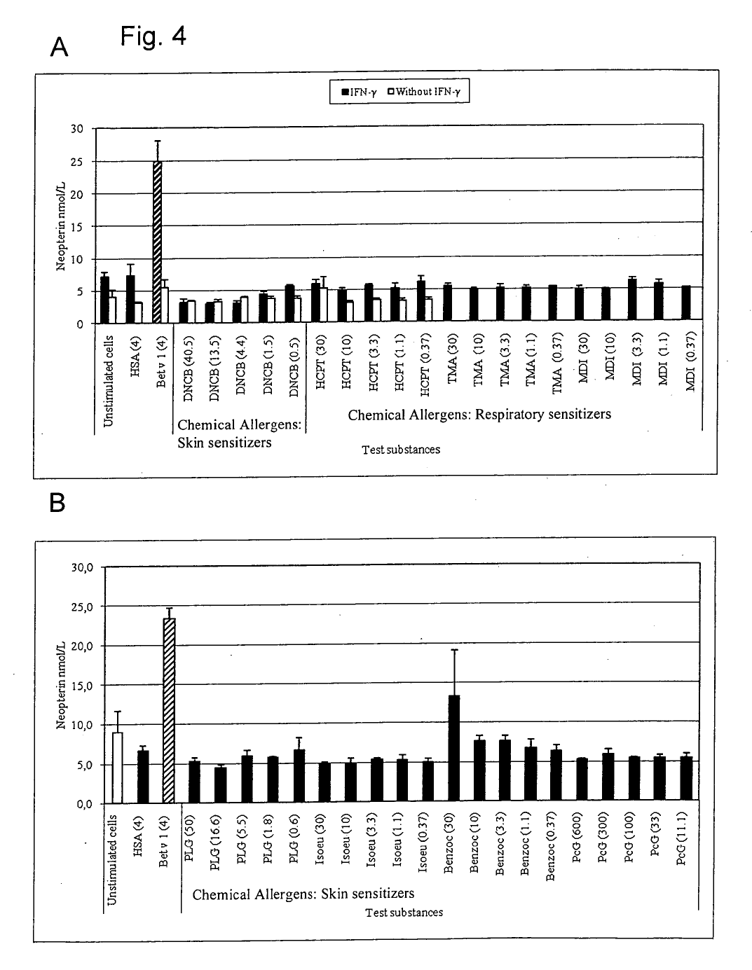

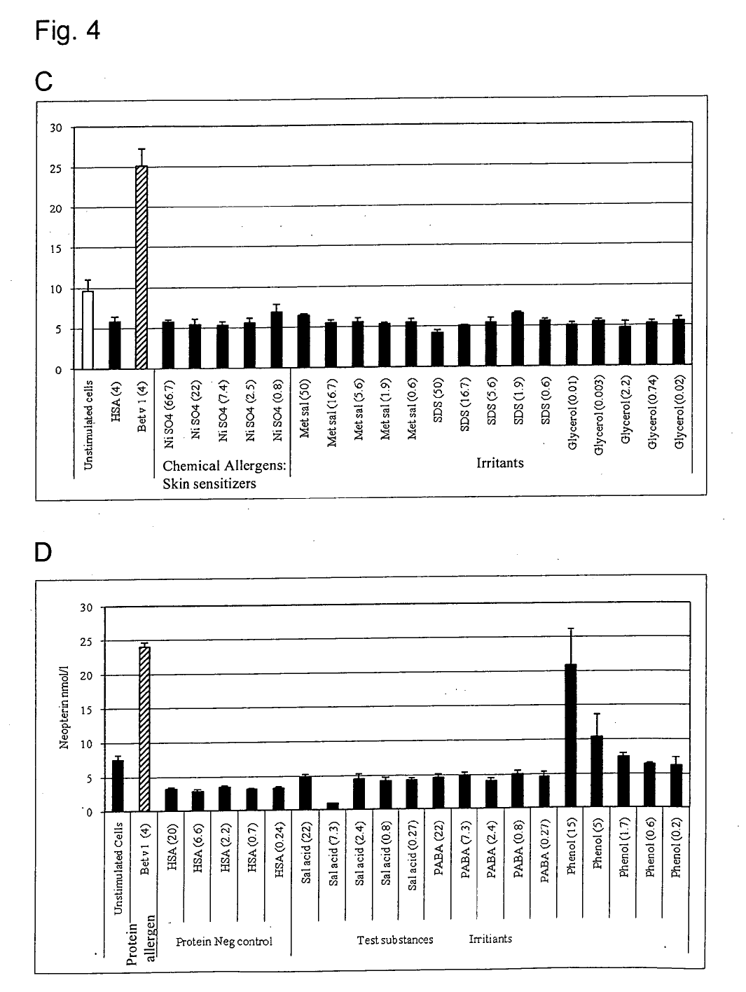

Low molecular chemicals are another group of substances that may induce hypersensitivity reactions, mainly skin- or respiratory sensitisations. In order to find out if there was a neopterin response towards these groups of substances, the test panel was increased to include some representatives from each chemical group as well as chemicals known to induce irritancy but not sensitization (FIG. 4a-d). First, the effect of IFN-γ on the neopterin release upon stimulation with one test substance from each category of sensitizers (DNCB, a strong skin sensitizers and HCPt, a strong respiratory sensitizer) as was evaluated (FIG. 4a). The results showed that none of the chemicals tested induced any neopterin release above background levels (unstimulated cells or cells stimulated with HSA), neither in the presence or absence of IFN-γ. Further assessment of known skin- or respiratory chemical sensitizers or irritants (tested at five different concentrations having approximately 80% of living cells at the highest concentration) showed that 2 out of 15 substances tested induced a neopterin response above background levels (FIG. 4b: benzocaine (30 μg/ml) and FIG. 4d: phenol (15 μg/ml)). The experiments were repeated at least twice with identical results.

### Further Optimization of Test Protocol: Cell Culture in Serum Free Media.

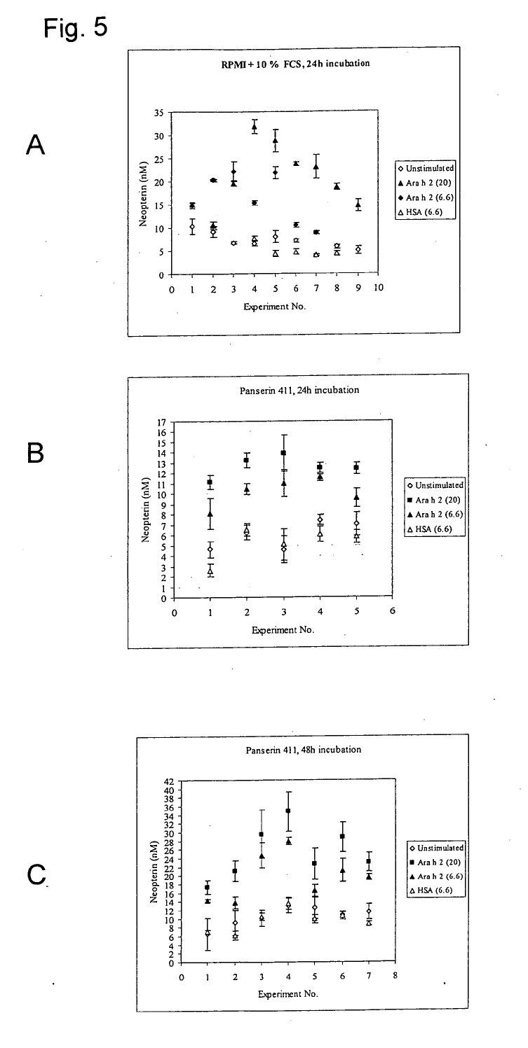

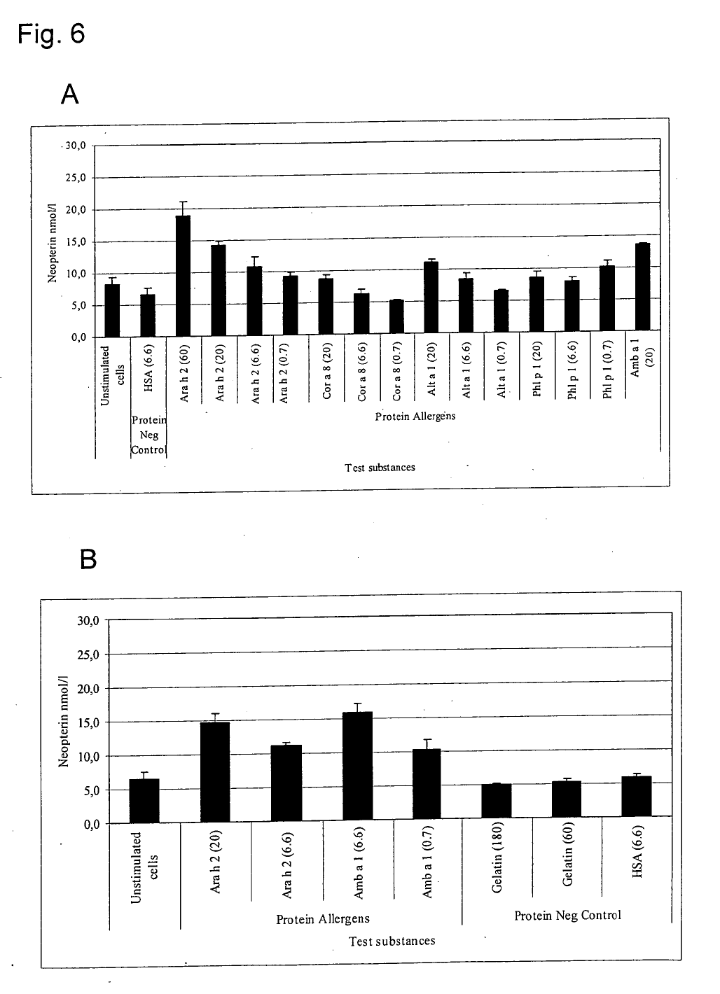

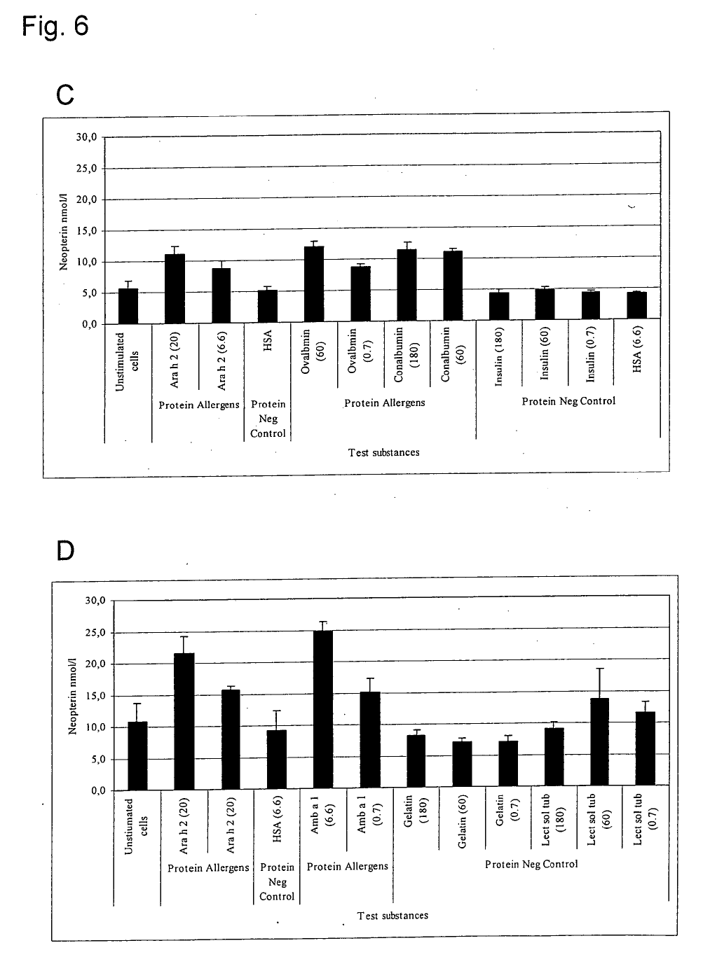

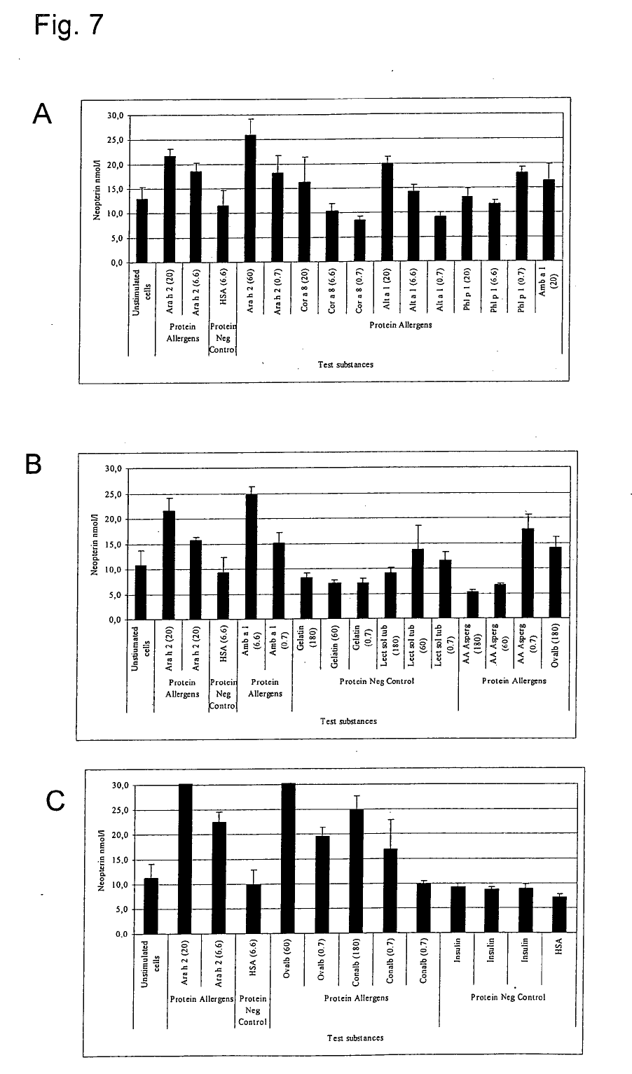

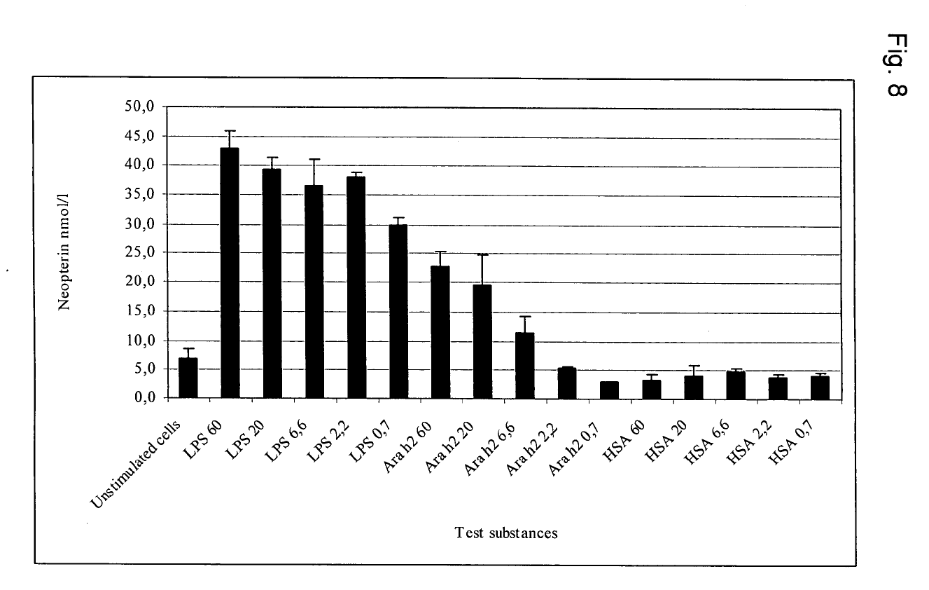

In order to have a more standardized test protocol lacking animal derived components, cells should preferably be cultured in media without serum (FCS or FBS). The MM6 were therefore adapted to serum free cell culture media (Panserin) and evaluated for its ability to respond to proteins allergens at different cell concentrations (Table 1) or different time points (24 hrs or 48 hrs) (Table 1, FIG. 5b-c), respectively. In Table 1, influence of cell concentration was determined. Due to a slightly higher neopterin response at 1×10⁶ cells/ml (cells cultured in Panserin), that cell concentration was chosen for further studies reported here, since the lack of serum proteins might require a higher cell density in order to secure a high cell viability. However, a cell concentration of 0.5×10⁶ cells/ml is still conceivable. Cells cultured in RPMI with 10% FCS (FIG. 5a) or in Panserin (FIG. 5b) were stimulated with Ara h 2 (20 or 6.6 μg/ml) or HSA (6.6 μg/ml) (in the presence of IFN-γ) for 24 hrs. At 24 hrs the allergen induced neopterin levels clearly distinct from those induced by the control protein for cells cultured in the presence or absence of foetal calf serum. The experiments were repeated nine times (FIG. 5a) or five times (FIG. 5b). At 48 hrs similar results were achieved (FIG. 5c) (experiment repeated seven times). These findings demonstrate that at 24 hrs or 48 hrs, cells cultured in serum free media respond by neopterin release in a similar way compared to what is archived when cells are cultured in RPMI for 24 hrs. Additional test substances (protein allergens) were tested with the serum free test protocol. Seven protein allergens and four proteins with no considerable allergenicity potential were tested at different concentrations for 24 hrs (FIG. 6a-d) or for 48 hrs (FIG. 7a-c). At both 24 and 48 hrs, the protein allergens induced a neopterin response in a dose response manner, similar to what was seen in cells cultured in RPMI supplemented with 10% FCS. However, in Panserin the levels of neopterin upon stimulation with protein allergens for 24 hrs are somewhat lower than what is seen in cells cultured in serum-containing media at same time point, however at 48 hrs, the neopterin levels in response to protein allergens are increased and similar to those levels seen in serum cultures cells stimulated for 24 hrs.

Evaluation of RPMI Versus Panserin: Statistic Evaluation of within Assay and Between Assay Precision

The within assay and between assay precision was evaluated for one native allergenic protein (Ara h 2; 20 and 6.6 microgram per ml) and for a non allergenic protein (HSA; 6.6 microgram per ml). In each experiment these two substances have been included on each cell culture plate during stimulation with test substances. For evaluation of between assay statistics, the results from one representative plate from each experiment were used. The results are shown in Table 2a-c. The cells were cultured in RPMI+10% FCS or in Panserin 411 and the test substances were stimulated with IFN-γ in the cell culture medium during stimulation. In each experiment the substances were stimulated in triplicate cultures at each concentration. The within assay (within triplicates) and between assay precision is shown in the table. The mean and standard deviation of the mean for triplicates in each experiment is indicated as well as the coefficient of variance (CV) within triplicates and between assays.

Outliers among data from cells stimulated with Ara h 2 and HSA and all test substances were defined if CV were ≧20%, if CV would be three times better and if the standard deviation were more than 1.5 nmol/l neopterin. At each experiment a number of wells with unstimulated cells were included and the results are also shown in the Table 2a-c. In experiments with 24 hrs stimulation the same criteria was used for definition of outliers among wells with unstimulated cells. In experiments where cells were stimulated for 48 hrs, one or two values out of 16 were withdrawn if they differed from the rest. Statistics were performed in Excel software. When cells were stimulated in RPMI+10% FCS for 24 hrs (Table 2a), the within assay CV was 10.3% at stimulation with HSA and even lower at stimulation with the Ara h 2 protein. The CV between assays were higher (between 20, 8% and 33.3%).

Also at stimulation in Panserin 411 for 24 hrs (Table 2b), the within assay CV was much lower (7.0%-16.7%) that between assays 14.2%-21.4%. The within assay CV was similar to that when cells were cultured with RPMI+10% FCS. The between assay CV was somewhat lower in the serum free media Panserin 411 and also the levels of induced neopterin were however lower in the absence of FCS. The levels of neopterin at stimulation with HSA were similar to the spontaneous neopterin production in unstimulated cells. Furthermore at stimulation in Panserin 411 for 48 hrs (Table 2c), the within assay as well as the between assay CV was higher at stimulation with Ara h 2. At stimulation with HSA, the within assay CV was slightly lower at 24 hrs compared to 48 hrs, the between assay CV was similar at 24 hrs and 48 hrs. The spontaneous neopterin production in wells with unstimulated cells showed a much variation at 24 hrs compared to after 48 hrs incubation.

Neopterin Levels were Increased Towards Protein Allergens in THP-1 Cells.

With the aim of elucidating whether another cell line with monocytic origin demonstrate the same cellular response towards protein allergens as MM6 does, the THP-1 cell line (Tsuchiya et al., 1980) was stimulated with a mini panel of test substances (different concentrations) including Ara h 2, LPS and HSA for 24 hrs (FIG. 8). The cells were cultured in RPMI supplemented with 10% FCS. The THP-1 responded with neopterin levels towards these substances similar to that seen with the MM6 cells. Dose response was seen for LPS and Ara h 2 but not for HSA (which did not exceed background levels). Further cell lines contemplated for use in the present invention are MUTZ-3, WBC264-9C and AML-193.

TABLE 1
Optimization of test protocol for cells cultured in serum-free media
(Panserin): Kinetics and cell concentration. MM6 cells were adapted
to Panserin media and stimulated with Ara h 2 for 24 or 48 hrs at
0.5 or 1.0 10⁶ cells/ml. Neopterin levels given in table.

| | conc | 0.5 milj | 1.0 milj |
|---|---|---|---|
| 24 h | | | |
| Ara h 2 | 20 | 11.1 | 13.9 |
| Ara h 2 | 6.6 | 11.1 | 11.0 |
| Unstimulated n = 12 | 0 | 5.4 | 4.6 |
| Unstim stdev | | 0.8 | 0.5 |
| Unstim CV | | 15.5 | 10.8 |
| 48 h | | | |
| Ara h 2 | 20 | 30.1 | 29.6 |
| Ara h 2 | 6.6 | 26.7 | 24.6 |
| Unstimulated n = 11 | 0 | 12.32 | 10.54 |
| Unstim stdev | | 1.9 | 1.3 |
| Unstim CV | | 15.8 | 12.3 |

TABLE 2a
MonoMac 6 cells were stimulated for 24 hrs with for the native peanut
allergen Ara h 2 (20 and 6.6 microgram per ml) and human serum
albumin (HSA; 6.6 microgram per ml). The substances were stimulated
with IFN-γ in the cell culture medium during stimulation. The cells
were cultured in RPMI + 10% FCS. In each experiment (assay) the
substances were stimulated in triplicate cultures at each concentration.
The within assay (within triplicates) and between assay precision is
shown in the table. The mean and standard deviation of the mean
for triplicates in each experiment is indicated as well as the
coefficient of variance (CV) within triplicates and between assays.
At each experiment a number of wells with unstimulated cells were
included and the results are also shown in the table.

| | Unstimulted | | Ara h 2 | Ara h 2 |
|---|---|---|---|---|
| Statistical analysis | cells | HSA | 20 μg/ml | 6.6 μg/ml |
| V(x′) | 2.5 | 1.9 | 48.0 | 29.0 |
| Mean of mean | 7.1 | 5.0 | 20.7 | 16.2 |
| Stdev of mean | 1.6 | 1.5 | 6.9 | 5.4 |
| Within assay CV (%) | 13.8% | 10.3% | 6.9% | 6.5% |
| Between assay CV (%) | 20.8% | 27.1% | 33.3% | 32.9% |
| Total CV (%) | 25.0% | 25.0% | 34% | 33.5% |

Unstimulted cells, n = 10
HSA (6.6 μg/ml), n = 6
Ara h 2 (20 μg/ml), n = 9
Ara h 2 (6.6 μg/ml), n = 7

TABLE 2b
MonoMac 6 cells were stimulated for 24 hrs with for the native peanut
allergen Ara h 2 (20 and 6.6 microgram per ml) and human serum
albumin (HSA; 6.6 microgram per ml). The substances were stimulated
with IFN-γ in the cell culture medium during stimulation. The cells
were cultured in the serum free media Panserin 411. In each
experiment (assay) the substances were stimulated in triplicate cultures
at each concentration. The within assay (within triplicates) and between
assay precision is shown in the table. The mean and standard deviation
of the mean for triplicates in each experiment is indicated as well as the
coefficient of variance (CV) within triplicates and between assays. At
each experiment a number of wells with unstimulated cells were
included and the results are also shown in the table.

| | Unstimulted | | Ara h 2 | Ara h 2 |
|---|---|---|---|---|
| | cells | HSA | 20 μg/ml | 6.6 μg/ml |
| Statistical analysis | n = 13 | n = 8 | n = 7 | n = 7 |
| V(x′) | 1.9 | 1.6 | 4.1 | 2.2 |
| Mean of mean | 6.3 | 5.4 | 12.0 | 9.7 |
| Stdev of mean | 1.4 | 1.3 | 2.0 | 1.5 |
| Within assay CV (%) | 13.7 | 16.7 | 7.0 | 10.4 |
| Between assay CV (%) | 20.4 | 21.4 | 16.5 | 14.2 |
| Total CV (%) | 24.6 | 27.1 | 17.9 | 17.6 |

Unstimulated cells, n = 13
HSA (6.6 μg/ml), n = 6
Ara h 2 (20 μg/ml), n = 7
Ara h 2 (6.6 μg/ml), n = 7

TABLE 2c
MonoMac 6 cells were stimulated for 48 hrs with for the native peanut
allergen Ara h 2 (20 and 6.6 microgram per ml) and human serum
albumin (HSA; 6.6 microgram per ml). The substances were stimulated
with IFN-γ in the cell culture medium during stimulation. The cells
were cultured in the serum free media Panserin 411. In each
experiment (assay) the substances were stimulated in triplicate
cultures at each concentration. The within assay (within triplicates) and
between assay precision is shown in the table. The mean and standard
deviation of the mean for triplicates in each experiment is indicated as
well as the coefficient of variance (CV) within triplicates and between
assays. At each experiment a number of wells with unstimulated cells
were included and the results are also shown in the table.

| | Unstimulted | | Ara h 2 | Ara h 2 |
|---|---|---|---|---|
| | cells | HSA | 20 μg/ml | 6.6 μg/ml |
| Statistical analysis | n = 7 | n = 6 | n = 7 | n = 8 |
| V(x′) | 4.9 | 6.0 | 35.7 | 28.9 |
| Mean of mean | 10.5 | 9.9 | 25.3 | 19.6 |
| Stdev of mean | 2.2 | 2.4 | 6.0 | 5.4 |
| Within assay CV (%) | 23.9 | 9.6 | 17.8 | 17.3 |
| Between assay CV (%) | 16.1 | 23.9 | 21.8 | 25.5 |
| Total CV (%) | 28.8 | 25.8 | 27.7 | 30.8 |

Unstimulted cells, n = 7
HSA (6.6 μg/ml), n = 6
Ara h 2 (20 μg/ml), n = 7
Ara h 2 (6.6 μg/ml), n = 8

1. A process for in vitro prediction of a potentially allergenic substance, characterized in that monocytes and/or macrophages and/or myelomonocytic cell lines are cultivated in the presence of the substance and interferon-γ, wherein the release of cytokines and/or neopterin is measured and an increase in release of cytokines and/or neopterin indicates that the substance is allergenic.

2. The process according to claim 1, characterized in that the release of neopterin is measured.

3. The process according to claim 1, characterized in that the presence or an increase of one or more cytokines chosen from IL-1, IL-1β, IL-2, IL-4, IL-5, IL-6, IL8 IL-10, IL-12, TNF-α and IFN-γ, is an indication a type I immediate hypersensitivity reaction from T and B lymphocytes and inflammatory cells and immediate type hypersensitivity such as asthma, hay fever, urticaria and rhinitis or an indication of class IV cell mediated T-cells immunity and delayed type hypersensitivity such as cellular immunity, delayed allergy and contact eczema

4. The process according to claim 1, characterized in that neopterin is measured as an indication of a type I immediate hyper sensitivity reaction.

5. The process according to claim 1, characterized in that the presence of neopterin is analysed, whereby the presence of neopterin is an indication of immediate type hypersensitivity such as asthma, hay fever and urticaria.

6. The process according to claim 1, characterized in that the analysis of IL-8 and neopterin is used to distinguish between class I and class IV cytokine profiles.

7. A process for in vitro prediction of a potentially allergenic substance, characterized in that monocytes and/or macrophages and/or
myelomonocytic cell lines are cultivated in the presence of the substance and interferon-γ, characterized in that the allergenicity of the substance is estimated by measuring up-regulated or down-regulated genes chosen from G1P2, OASL, IFIT1, TRIM22, IFI44L, MXI, RSAD2, IFIT3, IFITM 1, IFIT2, SPR, GNB2, C 33.28 HERV-H protein mRNA, IFITM3, XK, GPR15, MTIG, MT1B; MT1A, ADFP, IL8, MT1E, MT1F, MT1H, SLC30A1, SERPINB2, CD83, TncRNA or expression products from them are measured.

8. The process according to claim 7, characterized in that the expression of one or more of G1P2, OASL, IFIT1, TRIM22, IFI44L, MXI, RSAD2, IFIT3, IFITM1, IFIT 2, indicates Type I allergy; one or more of C 33.28 HERV-H protein mRNA, IFITM3, XK, GPR15, indicates TYPE I/IV haptenes and one or more of MT1G, MT1B; MT1A, ADFP, IL8, MT1E, MT1F, XK, IFITM3, MT1H, SLC30A1, SERPINB2, GNB2, MTIB, CD83, TncRNA genes indicates Type IV allergy.

9. The process according to claim 7, characterized in that RNA, DNA, amino acids, peptides or proteins are measured.

10. The process according to claim 1, characterized in that the substance to be tested is a protein.

11. The process according to claim 1, characterized in that interferon-γ is added before, simultaneously or after the addition of the substance to be tested.

12. The process according to claim 1, characterized in that the highest concentration of the substance being non toxic to the monocytes, macrophages and/or myelomonocytic cell lines is used.

13. The process according to claim 1, characterized in that the substance to be tested is serially diluted from the highest concentration of the substance being non toxic to the cells.

14. The process according to claim 1, characterized in that the cell line is selected from the group consisting of MonoMac-6, THP-I, MUTZ-3, WBC264-9C and AML-193.

15. A reagent kit for performing the process according to claim 1, characterized in that it comprises interferon-γ and test cells chosen from monocytes and/or macrophages and/or myelomonocytic cell lines.

16. The reagent kit according to claim 15, further comprising reagents which recognize cytokines preferably each of IL-8 and neopterin and/or reagents recognizing cytokine genes or up regulated genes.

17. The reagent kit according to claim 15, characterized in that the reagents recognize products produced during the expression of any of G1P2, OASL, IFIT1, TRIM22, IFI44L, MXI, RSAD2, IFIT3, IFITM1, IFIT2, SPR, GNB2, XK, IFITM3, C 33.28 HERV-H protein mRNA, IFITM3, XK, GPR15, MTIG, MT1B; MT1A, ADFP, IL8, MT1E, MT1F, MT1H, SLC30A1, SERPINB2, CD83, CD86, TncRNA.

18. The reagent kit according to claim 15, wherein the cell lines are selected from the group consisting of MonoMac-6, THP-I, MUTZ-3, WBC264-9C and AML-193.

19. The reagent kit according to claim 15, further comprising a cell culture medium suitable for culturing the cell line, antibiotics, a positive control, a negative control, a culture plate or flask, and/or instructions describing the method according to claim 1.

20. Use of a cell line in a process according to claim 1, characterized in that the cell line is selected from the group consisting of MonoMac-6, THP-1, MUTZ-3, WBC264-9C and AML-193.
